(19) United States
(12) Patent Application Publication
(10) Pub. No.: US 2022/0288809 A1
(43) Pub. Date: Sep. 15, 2022

(54) RADIO FREQUENCY TREATMENT TO PHYTOSANITIZE WOOD PACKAGING MATERIALS USED IN INTERNATIONAL SHIPPING

(71) Applicant: The Penn State Research Foundation, University Park, PA (US)

(72) Inventors: John J. Janowiak, Julian, PA (US); Karolina Szymona, University Park, PA (US); Kelli Hoover, Pennsylvania Furnace, PA (US); Ronald G. Mack, Chatham, MA (US); Mark Hamelin, Midland (CA)

(21) Appl. No.: 17/761,358

(22) PCT Filed: Sep. 29, 2020
(86) PCT No.: PCT/US2020/053249

§ 371 Date: Mar. 17, 2022

Related U.S. Application Data
(60) Provisional application No. 62/909,991, filed on Oct. 3, 2019.

(51) Int. Cl. B27K 5/00; A61L 2/08

(52) U.S. Cl. CPC B27K 5/0055; B27K 5/001; B27K 5/008; A61L 2/08; B27K 2240/15; A61L 2202/122

(57) ABSTRACT

A method for treating wood packaging materials using Radio Frequency heating includes the steps of heating wood packaging materials using RF heating and applying a pressure before the heating or incrementally applying a pressure during the heating. The wood packaging materials are heated in a RF operating unit that has a sealed chamber with an inner surface and a liner cover a majority of the inner surface, the liner having a heat-reflective inner face and an insulation layer between the inner face and the inner surface.

### REFERENCE TO RELATED APPLICATIONS

This application claims priority from Provisional Application No. 62/909,991 filed Oct. 3, 2019, the entire content of which is incorporated herein by reference.

### STATEMENT OF GOVERNMENT SUPPORT

This invention was made with government support under Grant No. 2018-51102-28338 and Hatch Act Project No. PEN04576 awarded by the United States Department of Agriculture. The Government has certain rights in the invention.

### FIELD OF THE INVENTION

The present invention relates to the use of radio frequency (RF) for the rapid phytosanitary treatment of commercial-sized loads of wood packaging materials, in a Radio Frequency (RF) treatment chamber during phytosanitary treatment of loads of Wood Packaging Materials (WPM) by applying RF dielectric heating and to a heat-reflective and insulating liner for the treatment chamber.

### BACKGROUND OF THE INVENTION

Wood packaging material (WPM; e.g. pallets, crates, and dunnage) is a vital part of global trade and the forest products industry. Pallets “move the world,” with several billion pallets used each day around the globe in domestic and international shipping. An estimated 50-80% of the US $12 trillion in world merchandise trade is moved using some form of WPM and more than 1.8 billion pallets are in service each day, and 93% of these are made from wood. In the U.S., roughly 700 million wooden pallets are produced per year. Untreated WPM is recognized as one of the major pathways by which wood boring insects and plant pathogens move among countries. In 2002, the International Plant Protection Convention (IPPC) established a requirement that all WPM be treated to reduce the risk of spread of quarantine pests. The International Standard of Phytosanitary Measures No. 15 (ISPM-15), adopted in 2014 by the IPPC of the UN after country consultation, mandated that all WPM used in international trade be treated by methyl bromide fumigation or conventional heat treatment to 56° C. at the core of the wood for 30 minutes.

Methyl bromide is a potential carcinogen and also classified as an ozone depleting gas with implications for global warming, which led to banning of this chemical in many countries. Methyl bromide is being phased out in the US and Europe (under the Montreal Protocol). Wood has inherently high insulation properties due to its cellular composition. Thus, the transfer of sufficient heat through wood to reach lethal temperatures for pests that infest the wood is slow using conventional heating. Conventional heating does not always kill all pests of concern. So the IPPC(International Plant Protection Committee—UN FAO) Secretariat put out a call for new treatments to be developed and submitted for approval to augment current ISPM-15 treatments.

With the addition of dielectric heating, e.g., RF and microwave (MW) to the approved treatments under ISPM-15, the treatment schedule requires that the wood temperature reach and hold 60° C., but the hold time at that temperature is only for 1 minute. Conventional heating under ISPM-15 requires a much longer 30-minute hold period once the WPM reaches a prescribed 56° C. core temperature and requires preheating of the oven.

MW also heats volumetrically by interacting with water molecules in the treated materials, but the frequency is much higher, ranging from 915 MHz to 2.45 GHz for most US commercial units e.g. heating oven applications. However, in direct contrast to MWs, RF dielectric applications use lower frequency irradiation with much longer wavelengths and thus can effectively penetrate materials more deeply allowing phytosanitation treatment of larger sections or volume of workloads of WPM.

In case wood is heated in an oven using any of the above-discussed methods, energy losses through the oven surface may render these methods inefficient and costly. Therefore, there is a need for a method or apparatus that prevents energy loss through the oven surface and makes these methods more efficient and cost effective.

### SUMMARY OF THE INVENTION

Dielectric heating occurs through two mechanisms: dipole rotation and ionic conduction. For RF, dipole rotation occurs when the material being treated contains polar molecules (positive and negative charges on opposite ends, like the water moisture within the wood), which subsequently align in the electrical field produced by dielectrically charged plates. The field alternates millions of times per second (1 MHz=1 million cycles per second), causing the polar molecules in the treated material to constantly rotate to align with the plates, producing friction that generates heat. In addition, charged particles (ions) in the material are heated constantly as they move to the opposite electromagnetic plate charge, adding more friction. These processes generate substantial kinetic energy (heat) that results in the whole volume of the product being heated at once, not just the surface, which is referred to as volumetric heating. As a result, the targeted WPM experiences rapid internal thermal heating in comparison to conventional or conductive heat transfer mechanisms.

RF does not require pre-heating and the chamber does not get hot during operation; most of the energy is directly absorbed by the product being heated rather than having to be transferred from the surface to the core of the product. RF can selectively heat insects over the product due to the higher water content of insects with respect to the product being treated (Nelson, S. O. 1996. Review and assessment of radio-frequency and microwave energy for stored-grain insect control. American Society of Agricultural Engineers 39(4):1475-1484).

In our experiments using RF to bulk treat raw wood to be used to construct crates and pallets, we found that substantial heating energy losses with a plateau or decline in temperature elevation occurs as the wood approaches or exceeds a critical temperature of approximately 50° C. This is due to water movement or vapor release during evaporative cooling, causing a non-steady heating unless significantly more power density is added in order to reach the required temperature of 60° C. through the profile of the materials being treated (per ISPM-15 schedule requirements). This WPM heating behavior causes both an increased treatment cost and an associated loss in ISPM-15 processing efficiency. Various techniques investigated include use of a thermal insulation barrier to contain heating losses resulting in some heating improvements but are not practical for large volume treatments.

The present invention provides a method in which heating behavior within large batches of WPM can be effectively controlled to reduce energy costs and increase treating capacity by applying a pressurization technique in conjunction with the operational functioning of the RF equipment. It was experimentally observed that adding controlled pressure levels of about 10-15 psi saved several hours of workload treatment time without having to increase the applied power density to satisfy the ISPM-15 treatment schedule.

In an embodiment of our invention, we have added a pressurization system to RF technology to allow WPM to reach the target temperature of the ISPM-15 schedule much faster. This approach works by maintaining a more constant heating rate during treatment and indirectly serves to better control temperature variations within the volumetric workload for purpose of an enhanced treatment quality control measure. In one version, the heating rate may be constant. In another version, a ramped heating rate may be applied. If the heating rate is constant, it is easier to monitor the process in terms of a predicted time to completion to reach a particular treatment time schedule. By minimizing thermal energy disparities within the wood load, greater heating uniformity can be achieved, which also avoids temperature extremes that otherwise can damage or degrade the WPM materials. As a result of the present invention, significant treatment cost savings can be realized by minimizing energy consumption and reducing moisture loss of the WPM, providing overall improvements in the processing efficiency while complying with ISPM-15 standard requirements.

The method may be carried by a RF operating unit, including a sealed chamber having two primary electrodes inside the chamber, i.e., a top electrode and a bottom ground electrode. A RF generator is connected to the electrodes for applying RF heating treatment to the WPM. A pressurization system is connected to the chamber for controlling the pressure inside the chamber. The system may typically include an infeed/outfeed track loader for simplification of loading and unloading the WPM workload, which reduces labor intensity. The pressure may be applied incrementally during the heating or applied fully before the heating cycle begins.

In some versions, the step of applying pressure to the chamber includes maintaining the chamber generally at a first pressure, such as approximately atmospheric pressure, during a first period and changing the pressure in the chamber generally to a second pressure after the first period. The second pressure may be at least 5 psi or at least 10 psi above atmospheric, such as approximately 15 psi above atmospheric. The first period may be defined by a passage of time or in terms of temperature of the WPMs. In one example, the first period is a time period that is predetermined based on the WPMs being treated. Alternatively, the first period may be defined as when the WPMs reach a threshold temperature. For example, the first period may end when at least some of the WPMs reach a threshold temperature in the range of 30 to 60° Celsius, such as approximately 50° C.

The temperature of the workload during the heating may be monitored using RF compatible temperature sensors placed within the workload or via an infrared (IR) surface scanning system to implement commercial quality control measures. In some versions, the “temperature of the WPMs” means an average temperature from the sensors or a maximum reading of any of the sensors or a minimum of any of the sensors. The temperature may also be inferred based on the passage of time, taking into consideration the type of WPM.

When the pressure is applied incrementally, the applying of the pressure step may include applying 5 psi of pressure before reaching a rise of 10° C. from an initial ambient temperature of the workload and adding another 5-10 psi to the chamber when 50° C. is first registered by a strategic placement of temperature sensors within the batch workload.

It is preferred that the wood not be heated to a temperature where curing occurs in terms of a significant moisture content loss; the WPM may remain near its original untreated condition or green state with moistures equal or near the fiber saturation level. It is preferred that the moisture content, after treatment, does not appreciably alter the characteristics of the WPM, such that mechanical properties (e.g., fastener installation and cant material resawing properties), are not substantially changed.

For this reason, it is preferred that the wood temperature stay below 100° C., and in some embodiments below 90° C., in further embodiments below 80° C., and, as stated above, typically temperatures below 70° C. are used. However, the temperature should nominally reach the prescribed 60° C. threshold to kill any life cycle pest infesting the WPM. It is preferred that the hold time is not longer than 2-5 at or above the prescribed 60° C. temperature elevation; however in some situations the hold time may be extended to as much as 30 minutes to assure treatment of all portions of the workload.

After reaching at least 60° C. with a 1-minute hold time, the chamber may be depressurized. The depressurizing of the chamber may be done at a constant rate. After the heating treatment and depressurization, the workload may be removed from the chamber for cooling and post-treatment construction of shipping materials.

The surface temperature of the workload may be further checked using surface temperature imaging technology after the depressurization step to further verify that adequate phyosanitation treatment was achieved in compliance with ISPM-15.

Our preliminary experiments using the method of the present invention in RF processing technology showed a reduction in moisture losses within the batch of treated materials to help avoid drying-related wood surface checking defects. We also saw reduced evaporative cooling, which is a process that significantly increases the time (and energy input) required to reach lethal temperatures to kill all pests infesting the wood being treated.

Our research on both MW and RF and interactions with the industry have clearly shown that RF is far more likely to be adopted than MW because of its greater depth of electromagnetic field wave penetration and ability to bulk treat WPM, which is something MW cannot do under normal operational or application circumstances (Dubey et. al. 2016).

Certain embodiments of the present invention may have three very significant benefits: 1) it keeps electrical power consumption to a minimum, thereby reducing operational energy costs; 2) allows for greater processing efficiency, which will increase capacity for the company, producing a higher return on the capital investment in the equipment; and 3) RF is a more environmentally friendly replacement to methyl bromide fumigation and conventional heating, producing lower carbon emissions as the industry seeks to comply with ISPM-15 to reduce risks of movement of pests in WPM used in international shipping (and now domestic shipping as well with new rules).

This technology could be applied to not only effectively treat WPM but it would also benefit RF treating schedules used for other commodities such as phytosanitation of sawn timbers used extensively in timber frame construction, for either domestic or imported products. In addition, this innovation could be equally applied to round wood sections, such as export wooden sawbolts (sawlogs) or other export commodities. For example this innovation is applicable to control the desired temperature elevation for phytosanitary workloads involving RF treatment of wood chips (domestic use or for export), where the heat dissipation factor via water evaporative cooling effects is enhanced due to increased wood surface area that permits greater losses of stored thermal energy.

Heat can be transferred by conduction, convection and by radiation. Conduction requires direct contact with the heated surface (e.g. air conveys energy from heated wood and then heated air passes the energy to the steel cylinder). It is a mechanism of passing the heat directly from a warmer mass to a cooler surface. Convection spreads heat within fluids e.g. water or water vapor, when molecules of the liquid or gas are moving relatively freely. Convection streams occur in cases of unequal heating. When air containing water vapor in an RF chamber warms up, it will expand and its density decreases in comparison to the air above, which will cause air temperature to rise. When air cools, it becomes denser and it sinks. Another form of heat transfer is radiation. Thermal radiation is generated by the emission of electromagnetic waves. Those waves are a result of random movements of charged electrons and protons within the matter in the WPM treatment chamber. All materials that have a temperature above absolute zero effectively radiate some amount of electromagnetic radiation generated by heat. Radiation can be described as the exchange of energy by photons; hence, unlike convection and conduction, it does not require a medium, and it occurs even in a vacuum. Electromagnetic radiation travels at the speed of light (as radiant heat travels from the sun to the Earth) and is either transmitted through, absorbed into, or reflected by, any material it contacts. The hotter the object, the more heat it radiates.

Therefore, in an embodiment according to this disclosure, a RF treatment chamber such as a steel vessel, has a liner covering a majority of the inner surface of the chamber. The liner includes a heat-reflective inner face and an insulation layer between the inner face and the inner surface of the chamber. The layer reflects the thermal radiation from the chamber walls back towards the heated wood material and the insulation helps to contain the thermal energy. The inner walls define an inner surface of the conductive steel vessel.

An embodiment of a method of treating wood packaging materials (WPMs) using Radio Frequency heating according to this disclosure comprises the step of: providing a RF operating unit. The RF operating unit has a sealeable chamber having an inner surface, a liner covering a majority of the inner surface, a RF generator connected to the chamber for applying RF electromagnetic energy treatment to the WPM, and a pressurization system for controlling the pressure inside the chamber. The liner has a heat-reflective inner face and an insulation layer between the inner face and the inner surface of the sealable chamber. The method also include the steps of loading the chamber with a workload of the WPMs, applying an above-atmospheric pressure to the chamber during the treatment; treating the WPMs using RF heating until a temperature of the WPMs reaches a predetermined temperature of generally not more than 100° C.; and maintaining the predetermined temperature for at least 1 minute.

In some examples, the liner covers at least 75% of the entire inner surface of the chamber. The heat-reflective inner face may be aluminum foil, metallic aluminum or aluminum anodized fabric, such as aluminum anodized polyester fabric, and may have a heat reflectivity of at least 90% or at least 95%. The heat reflectivity may be measured in the infrared range. A less preferred option is to apply heat-reflective paint to the inner surface of the chamber, either with or without an insulation layer. In some examples, heat-reflective paint may include titanium pigment, aluminum pigments and/or ceramic bubbles. In some examples, heat-reflective paint is applied to portions of the inner surface where the combination of aluminum foil/fabric and insulation is difficult to apply or less necessary. For example, paint may be applied under the area at the bottom of the chamber, which may be occupied or covered by a material support during use. n some examples, the liner can withstand a temperature up to 90° C., 150° C., or 250° C.

In some examples, the heat-reflective inner face is formed of a material that is moisture impermeable, which may be defined as having a permeability rating of 0.1 perm or less.

As mentioned, the insulation layer is disposed between the heat-reflective inner face and the inner surface of the chamber. In some examples, the insulation layer is silicone foam or polyamide foam having an thermal conductivity of less than 0.07 W/(m·K) or less than 0.05 W/(m·K). The liner may have an acid tolerance to a pH level or 3.0 or of 3.5. The insulation layer may not absorb much moisture, such as not absorbing more than 5 percent of its weight in moisture when exposed to operating conditions for 24 hours.

In some examples, the liner is installed such that there is substantially no air gap between an outer surface of the insulation layer the inner surface of the chamber.

In some examples, the insulation layer has a moisture retention of less than 5% weight gain when exposed to moisture for a 24 hour within a repeated to continuous cylinder treating period.

In some examples, an insulation layer is placed on top of or underneath the WPM to help retain a spontaneous heating response. This insulation layer may be 100% wool e.g natural keratin fiber as woven fabric having a thickness of at least 0.1 inch.

In some embodiments, the predetermined temperature during treatment is not less than 60° C., and/or the predetermined temperature is not more than a maximum average temperature of 90° C., 80° C. or 70° C. with the aggregated SWPM. The predetermined temperature may be maintained not longer than a period of 5 minutes, 4 minutes, 3 minutes or 2 minutes.

In some embodiments, the step of applying of the pressure to the chamber includes maintaining the chamber generally at a first pressure during a first period and changing the pressure in the chamber generally to a second pressure after the first period. The first pressure may further be approximately atmospheric pressure and the second pressure greater than atmospheric pressure. In some embodiments, the first period is defined by elapsed time, and the elapsed time period is predefined based on the WPMs being treated. In other embodiments, the first period is defined by a temperature threshold, and the first period ends when the temperature of at least some of the WPMs as the volumetric load reaches a temperature threshold in the range of approximately 30° C. to approximately 60° C.

In alternate embodiments, the method further includes depressurizing the chamber after reaching at least 60° C. with a 1-minute hold time. In some embodiments, the depressurizing of the chamber is at a rate of decreased pressure of 2-4 psi per minute. In other embodiments, the heating treatment is at a constant rate or at a ramping rate.

Adding an insulation component to the cylinder helps to preserve the remaining energy that is not reflected or transferred by conduction or convection. Hence, the combination of these two component properties work in a complementary manner. Thermal conductivity K can be described as the ability of heat to pass from one side of a material through to the other and this is expressed in a given unit (W/(m·K) in International System of Units or Btu/(h·ft·° F.) in imperial units). The lower the thermal conductivity of the material, the better the insulation properties. The R-value (K·m²/W) is the factor that indicates the resistance of the material in conducting heat, so the higher the value of R, the greater the insulation. R-value relates to the thermal conductivity of a material used as an insulation, and the functionality inversely corresponds to an effective thickness.

$$\frac{1}{R} = \frac{K}{L}$$

Where R is the R-value across the thickness of the liner layering, K is the material's coefficient of thermal conductivity and L is the given thickness as an insulating property barrier.

During dielectric phytosanitary treatment, WPM typically experiences rapid internalized as a spontaneous thermal heating event in comparison to conventional or conductive heat transfer mechanisms. Although most of the electromagnetic energy is directly generated within the product being heated rather than having to be transferred from the surface to the core of the product, some heating mechanism energy is ultimately absorbed by the chamber vessel. In our experiments using RF, which involve bulk treating raw wood to be used to build product crating and shipping pallets, we found that using a suitable reflective/insulation liner as a thermal barrier applied to the RF kiln treatment chamber prevents or mitigates the passive heat movement and acts to control thermal energy radiation losses from the treated wood commodity. The term reflective/insulation liner as used in this disclosure may have a reflective layer, an insulation layer or a combination of the reflective and insulation layer installed inside the RF chamber that covers an inner surface of the RF chamber. Without a reflective/insulation liner, this thermal energy from the workload is transferred or lost to the highly conductive steel material of the dielectric field treating chamber where this subsequent heat accumulation then dissipates from the treating vessel to the surrounding environment.

FIGS. 1A-1C are thermal images of outer surface of the RF chamber during the phytosanitation treatment of wood components. FIG. 1A is a thermal image of the RF chamber without a reflective/insulation liner installed on the inner surface, while FIG. 1B is a thermal image of the RF chamber that has reflective/insulation liner installed partially covering the inner surface. The darker surface areas in FIG. 1B indicate lower heat dissipation through the outer surface of the RF chamber, while the lighter surface areas indicate higher heat dissipation. It should be noted that the surface of the RF chamber where the reflective/insulation liner is installed shows darker surface areas in FIG. 1B. FIG. 1C is a thermal image showing heat dissipation “leakage” from seams of the reflective/insulation liner that is installed on the inner surface of the RF chamber and shows a lighter surface area indicating higher heat dissipation surrounded by darker surface areas indicating lower heat dissipation through the outer surface of the RF chamber.

### BRIEF DESCRIPTION OF THE DRAWINGS

The accompanying drawings, which are included to provide a further understanding of the disclosure and are incorporated in and constitute a part of this application, illustrate embodiments of the disclosure and together with the description serve to explain the principle of the disclosure. In the drawings:

FIGS. 1A-1C are thermal images of the outer surface of the RF chamber during the phytosanitation treatment process;

FIGS. 2A and 2B are post-treatment thermal images of wood cants/wood stringers located inside the RF chamber following the completed dielectric heat treatment;

FIG. 3A is an drawing of the RF chamber covered with reflective liners as applied to act as a thermal barrier;

FIG. 3B is a thermal image of reflective liners after a completed RF phytosanitation treatment as an investigation to study heat loss behavior from the RF chamber.

FIG. 4A is a temperature verses time graph for a RF chamber having one layer of heat-reflective fabric on the inner with respect the highly conductive chamber material surface;

FIG. 4B is a temperature verses time graph for a RF chamber having double sided aluminum foil with bubble plastic core as a reflective layer on the inner surface investigated to control systematic occurrences of treating heat dissipation;

FIG. 4C is a temperature verses time graph for a RF chamber having no liner application as thermal barrier on the inner steel cylinder surface;

FIG. 5A is a temperature versus time graph for the outer surface of the RF chamber to monitor conductive heat exchange;

FIG. 5B is a temperature versus time graph for the inner surface of the RF chamber to comparatively examine the enhancement e.g. improved heat retentions within the treatment chamber;

FIG. 6 is a temperature versus time graph recorded during RF treatment of white ash decking boards inside the steel cylinder vessel;

FIG. 7A is an image of a high R-value silicone foam as a reflective/insulation liner applied as an experimental thermal test barrier installed inside the RF chamber;

FIG. 7B is a thermal image of the RF chamber during phytosanitation treatment;

FIG. 8 is another graph of the temperatures recorded during RF treatment of Yellow Poplar pallet construction stringers;

FIG. 9A is an image showing casting of Casting Sicomin® PB 250 DM 02 (epoxide expanding foam) on a steel panel segment for experimental as a thermal barrier investigation;

FIG. 9B is an image showing the cured with closed cell rigid epoxy foam casted on the steel panel;

FIG. 9C is an image showing testing samples in various temperature conditions in a high temperature ceramic furnace to test potential debonding problem with respect heating responses as the thermal expansion of the conductive metal;

FIG. 9D is an image showing samples after thermal treatment for different timings;

FIGS. 10A-10B show images of Manton cork after the RF treatment;

FIGS. 11A-11C show images of a FOAMULAR® 250 rigid extruded polystyrene foam board being used as a wood load insulation;

FIG. 12A is an image of a Rothco® wool blanket application around some of the Eastern White Pine sawlogs;

FIG. 12B is a thermal image of Rothco® wool blanket wrapped around some of the Eastern White Pine logs during the applied RF energy for spontaneous dielectric heat treatment;

FIG. 13 is a schematic front view of an exemplary embodiment used during pytosantitation with the chamber door shown opened for track loading or unloading of the volumetric batch of SWPM;

FIG. 14 is a cross-sectional side view of an exemplary chamber used for phytosanitation as a prototype equipment design for potential commercial end-users;

FIG. 15 is an exploded view of detail A shown in FIG. 14; and

FIG. 16 is a flow chart showing a systematic batch process for pressurization with RF volumetric heat treatment to sanitize WPM in accordance with one embodiment of the present disclosure.

### DETAILED DESCRIPTION OF THE INVENTION

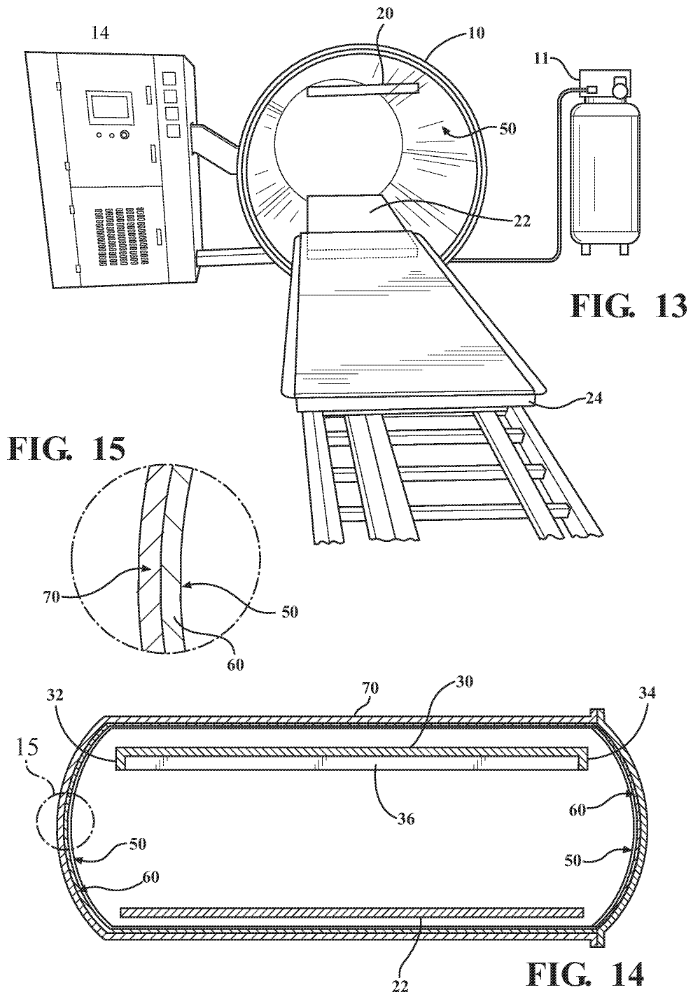

FIG. 13 is a schematic view of an exemplary layout of a radio frequency system for RF dielectric treatment of the wood packaging materials (WPMs). In one embodiment, as shown in FIG. 13, the system arrangement includes a sealable chamber 10, used as a pressurization treating cylinder or treatment retort, a pressurization system 11, e.g. an air supply pump for retort pressurization, a RF operational cooling system, a RF (3-30 MHz frequency) electromagnetic input power generator (oscillator or other) 14, with a suitable integrated PLC control system as the functional mechanism for applied power density to regulate targeted WPM heating rates, and an infeed/outfeed track loader 24 for loading and unloading of the workload. Overall, the cooling system of higher power RF heating units must be suited for rapid cycle sanitization, e.g., those that run with applied operational power below 30-50 kW, which may optionally include only an air-induction fan system for cooling to dissipate excess RF tube heat.

The chamber 10 shown in the center region of the layout may be an adequate construction cylinder or box-shaped design. In one example, the chamber 10 is the type of chambers used for vacuum with moisture drying treatments of wood, in the form of sawn lumber and timbers. The present design was specifically modified to allow or enable chamber retort pressurization. The chamber 10 can include either a manual or hydraulic sealable door 18 which can be freely swung open or closed to facilitate loading/unloading the volumetric batches of WPM.

The chamber 10 includes two primary electrodes including a retractable compression electrode plate 20 as the top electrode and a ground electrode 22. The retractable compression top electrode plate 20 is lowered or retracted by air cylinders between the loading and the unloading of the workload. The bottom ground electrode is in a position fixed inside the lower portion of the retort 10. As the workload is fed into the chamber 10 by the infeed/outfeed track as the workload transport loader, the volumetric workload is placed on the transport table 24 and positioned between the top electrode 20 and the bottom ground electrode 22. The top electrode applies a download load pressing down onto the workload to reduce air gaps between the top electrode and the lower ground electrode.

Additional secondary electrodes may be used to improve the energy field distribution depending on the depth of the workload. Secondary electrodes may be statically placed between the built up rows of WPM to be treated and applied as a batch treatment. The secondary electrodes are manually removed after the workload is effectively removed from the cylinder. In an alternative embodiment, instead of secondary electrodes, the top flat electrode may be modified with a winged electrode design arrangement. The top flat electrode 30 may include electrode plate wings, e.g., along the entire perimeter of the flat electrode plate 30, including two ends and two sides. FIG. 14 is a cross-sectional view showing three secondary electrodes 32, 34, 36 attached to the flat electrode plate 30, one at each end and one of the parallel sides of the electrode 30, facing the bottom ground electrode 22.

The primary electrode pair or secondary electrodes are connected to the RF power input generator 14. The RF generator 14 supplies an alternating current to introduce an electromagnetic field. In one embodiment, the RF generator has a constant or variable power output of 50 kW or with greater heating rate capacities. In one embodiment, an operational electromagnetic dielectric frequency may be in the range of 5 to 30 MHz or other wavelength frequency suitable to achieve the desired depth of penetration for wave energy adsorption to obtain heating uniformity during dielectric electromagnetic treatment of an entire WPM volume. The pressurization system 12 provides systematic pressurization of the chamber during the active RF treatment. Just as water evaporates at a higher temperature under an air pressure higher than atmospheric, the pressurization technique of the present invention helps to prevent moisture and significant thermal heat energy losses during the phytosanitary heating cycle by RF treatment to more rapidly and cost effectively comply with ISPM treating requirements.

The temperature within the workload may be monitored throughout the treatment. The temperature monitoring may be done by factory-calibrated fiber-optic or other RF compatible temperature sensors. An access port (not shown) on one side of the retort enables running (routing) of the required fiber-optic sensors inside the retort and continuous monitoring of the workload heating coupled to an independent data collection system.

Some exemplary dimensions of a system in accordance with the present invention are as follows. In one embodiment, the chamber measures 3-m×1-m×1-m. The volume capacity to be heated as shown is equal to ˜3 cubic meters, although greater capacity workload designs may be built for large-scale commercial treaters. The electrode plates measure roughly 3-m×1-m. The infeed/outfeed track loader measures 4-m×1-m.

An important component of the RF system innovation includes adequate positive pressure control to raise the boiling point of water or otherwise control the conversion of liquid moisture content to a gaseous water vapor phase that results in net moisture content reduction, while also preventing the critical losses of thermal energy needed to rapidly, and with desired uniformity, elevate the WPM temperatures throughout the bulk volume of the treated load.

The present invention provides a method of treating WPM to eradicate invasive pest organisms using otherwise a conventional RF oven or vacuum operated kiln type of dielectric dryer technology.

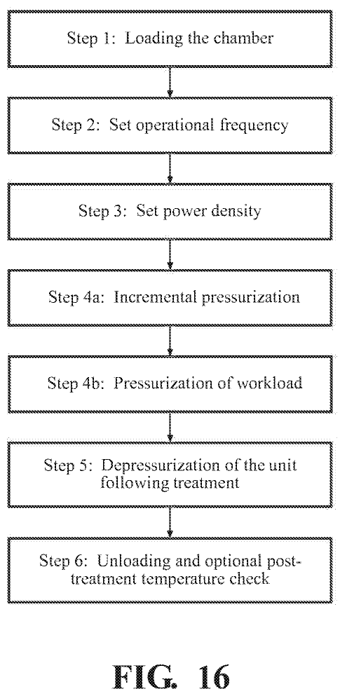

FIG. 16 is a flow chart showing a systematic batch process for pressurization with RF volumetric heat treatment to sanitize WPMs infested with wood pests in compliance with approved ISPM 15 in accordance with one embodiment of the present invention. Each step will be elaborated as follows.

Step 1. Loading the chamber:

Fill the RF operating unit cylinder (Pressure Design Retort) with the WPM Volumetric Load. In some embodiments, the RF operating unit cylinder has a reflective/insulation liner covering the inner surface of the RF cylinder.

The volumetric load may be defined as multiple sawn dimension 4″×6″ cants (hardwood/softwood) or other sized raw material pieces to be batch treated prior to conversion into wooden shipping pallets or as otherwise utilized as dunnage for domestic/international commerce.

The unit must be equipped with suitable electrodes (electromagnetic applicators as the field intensity guides) to assure compliance with the ISPM-15 treatment schedule for Dielectric Heating (DH), i.e., hold temperature of not less than 60° C. for 1 min through the profile of the workload.

Temperature process monitoring may include factory calibrated fiber-optic or other RF compatible temperature sensors with strategic placement within the workload, consistent with the ISPM-15 standard requirements to monitor heat elevation and uniformity of heating throughout the workload.

Step 2. Set operational frequency:

The next step is to secure the unit retort loading door and apply the appropriate alternating dielectric RF electromagnetic field (EMF). Typical operational frequency is 4 to 50 Hz (EMF oscillations per second).

The appropriate dielectric field will vary as a function of the energy delivered to the targeted workload depth where an ideal frequency is verified based on known or approximated dielectric properties of the WPM, which can vary by wood species and inherent wood moisture content (% MC).

Step 3. Set power density:

Treatment field intensity or application power density vary depending on rated RF generator capacity.

The power density will vary based on the selected RF equipment where higher-power rated designs will increase the processing capacity for a commercial ISPM-15 certified treating facility. Optimum RF heating power relative to pressurization is a function of the combined interactions of material density with weighted % MC, wood species permeability, and ambient thermal state of the volumetric batch of the SWP to be treated.

Power density is calculated based on the desired treatment schedule (treatment time, workload size, wood species and moisture content considerations) to be in compliance with ISPM-15. Anticipated operational power density is 2-4 kW/m³ or higher depending on operational treating to RF generator capacity.

Step 4a. Incremental Pressurization:

The step of incremental pressurization includes a) applying 5 psi of pressure before reaching a rise of 10° C. from the initial ambient temperature of the workload and b) adding another 5-10 psi to the chamber when 50° C. is first registered by a temperature sensor within the workload.

From experimental results conducted on ash (Fraxinus spp.) cants (green SWP measured at or above the fiber saturation point, e.g. >30% wood moisture content), the combination of applied power density (maximum 3.3 kW/m³) and 10 psi pressurization was shown to substantially reduce the total batch treatment time to fully comply with ISPM-15 requirements (60° C. with 1-minute temperature hold), while reducing the required energy consumption, thereby achieving significant operational cost savings.

Step 4b. Pressurization of workload:

Typical starting pressure recommended is in the range of 10-20 lbs per square inch (psi). Higher pressure can be considered as an option to achieve further batch heating uniformity based on observed departure from a constant workload heating rate to minimize treatment duration.

An alternative approach to incremental pressurization may be used where full pressurization is applied before initiating the RF heating cycle.

Step 5. Depressurization of the unit following treatment:

Depressurization should be controlled for a slow release of pressure. Pressure reduction should be applied only after reaching 60° C. with a 1-minute hold time as required by ISPM-15. A rate of decreased pressure of 2-4 psi per post treatment minute is recommended.

Step 6. Unloading and optional post-treatment temperature check:

An optional step following decompression is to check surface workload temperatures using surface temperature imaging technology, such as IR. Then open the unit door and remove the workload to verify ISPM-15 compliance. The workload is removed for cooling and post-treatment construction of shipping materials.

During this RF treating process, RF heating is applied to the WPMs while a pressure is added to the chamber, until the WPMs are heated to a temperature of about 60° C., but preferably less than 90° C., for a hold time from 60 sec to a few minutes. Under this operating condition, the moisture inside the WPMs is mostly preserved. It is preferred that the wood not be heated to a temperature where curing occurs in terms of a significant moisture content loss where the WPM may remain near its original untreated condition or green state with moistures equal or near the fiber saturation level. For this reason, it is preferred that the wood temperature stay below 100° C., and in some embodiments below 90° C., in further embodiments below 80° C., and, as stated above, typically temperatures below 70° C. are used. However, the temperature should nominally reach the prescribed 60° C. threshold to kill any life cycle pest infesting the WPM. Pressures in the range of 10-20 psi are preferred, with 15 psi being typical. It is preferred that the hold time is not longer than 5 minutes at or above 60° C., in some embodiments not longer than 4 minutes, and in further embodiments not longer than 3 minutes, and in still further embodiments not longer than 2 minutes. As noted above, it is preferred that the moisture inside the wood is mostly maintained for ease of post-treatment conversion to wooden constructed shipping pallets or other packaging end-use applications. In some embodiments, this means that the moisture content, after treatment, remains in the original range of wood fiber saturation typically 28 to 30% MC and in further embodiments it means that the moisture content is not reduced by more than a few percentage of the original wood moisture content. For some embodiments, it is preferred that the moisture content of the wood averages (some pieces may be drier and some may be wetter) at least approximately 28% before the process starts.

In an alternative process, the step of applying pressure to the chamber includes maintaining the chamber generally at a first pressure, such as approximately atmospheric pressure, during a first period and changing the pressure in the chamber generally to a second pressure after the first period. The second pressure may be at least 10 psi above atmospheric, such as approximately 15 psi above atmospheric. The first period may be defined by a passage of time or in terms of temperature of the WPMs. In one example, the first period is a time period that is predetermined based on the WPMs being treated. Alternatively, the first period may be defined as when the WPMs reach a threshold temperature. For example, the first period may end when at least some of the WPMs reach a threshold temperature in the range of 30 to 60° C., such as approximately 50° C. The temperature of the WPMs may be an average temperature from the sensors or a maximum reading of any of the sensors or a minimum of any of the sensors.

It may be preferred to not apply pressure until after a period of time or until a temperature increase is made. This allows moisture from an inner part of a load of WPMs to migrate to the surface, thereby allowing more even heating of the load of WPMs. It may also be preferred that the load of WPMs is arranged such that air gaps are reduced, and a load may be applied vertically and/or horizontally to reduce the air gaps. In one example, the WPMs are randomized or rearranged such that portions that were outside in a bundle are now inside and vice versa. The wood pieces may also be cut from the as-received size prior to treatment and then re-stacked. The use of thinner or smaller wood pieces allows for reduced air gaps since the thinner or smaller pieces will deform under a load during treatment more easily than larger pieces. According to an alternative embodiment, wood chips may be treated and be considered as the WPM.

Experiments were conducted to monitor the temperature rise in wood samples being treated without the application of pressure. It was found that some portions of the load heat very quickly and reach a temperature of 100° C. or above while other portions of the load heat very slowly. In this test, it is believed that the lowest temperature reading may be an error. However, even if this data is ignored, it still took approximately 280 minutes for most of the load to reach 60° C. As noted, the moisture content dropped by 6.45 percent. Another experiment was conducted to monitor the temperature rise in wood samples being treated with the application of pressure after a period of time has elapsed. Specifically, the chamber was maintained at approximately atmospheric pressure for approximately 70 minutes. The term “approximately atmospheric pressure” is used herein to indicate that additional pressure is not applied. However, some pressure increase may occur due to the heating of the chamber. At the point where at least some of the WPMs reach a threshold temperature of 50° C., the pressure is increased to approximately 15 psi above atmospheric. As observed, the temperature readings in the chamber remained closely grouped and all readings (save for the erroneous lowest reading) reach a treatment temperature of 60° C. after approximately 150 minutes, at which point no readings are at 100° C. The treatment time is dramatically reduced, and the moisture content loss is only 4.16 percent.

In one embodiment, the system has a 3-phase electrical source of 480 or optional 600 volts and a total input power of 125-150 amps at 480 volts, supplied by the service alternating current (voltage with power input) transformer.

In one embodiment, the system includes a cooling system having a cooling capacity of 159960 kcal/h or higher. The cooling system may be an evaporative cooling system comprised of stainless steel cabinets, heat exchangers, water circulation pumps and exhaust fans.

In one version, the system includes a fully-automated control system having touch screen controls. The control system is operable to perform temperature monitoring and control, moisture content monitoring and control, cooling system monitoring and control, and pressure monitoring and control.

In one version, when fully assembled and before the infeed cart is fed into the chamber, the footprint of the equipment is about 12 m L×4.3 m W×2.63 m H.

Installing a reflective/insulation liner helps prevent thermal energy losses during the phytosanitary heating cycle by RF treatment and enables more rapid and cost-effective compliance with the ISPM-15 schedule for WPM. This disclosure provides a method in which heat utilized within large batches of WPM can be effectively preserved in the vessel by applying an appropriate liner material in conjunction with the operation of the RF equipment.

In an embodiment of the disclosure, a suitable reflective liner system is added to the RF technology to allow the WPM to reach the target temperature of the ISPM-15 schedule faster. This provides two very significant benefits: 1) It serves to keep electrical power consumption to a minimum, thereby reducing operational electrical energy costs; and 2) Allows for greater RF treating process efficiency, which will increase output capacity for a manufacturer, producing a higher net return on the capital investment in the equipment technology. This disclosure could prove even more beneficial to control the desired temperature elevation for phytosanitary workloads.

Heating of the RF chamber walls during treatment occurs because of the combination of pressurization with randomized moisture content (hereafter referred to as RFP); treating stacked, constructed pallet components produces less air space between wood pieces, especially when treating larger arrays of wood. These factors redistribute the wood dielectric properties within the bulk volume causing more intense RF electromagnetic field (EMF) interactions. The added pressure keeps the moisture in the workload, which heats more quickly but does not control surplus thermal energy radiations that are emitted, heating the conductive metal of the RF chamber enclosure. In another words, using RFP during the treatment cycle keeps significant amounts of water vapor from escaping the wood material (evaporation heat releases), but does not completely prevent thermal radiation losses.

Various techniques were investigated to provide a thermal insulation barrier to contain observed heating losses, in order to improve upon the required heating schedule. In experiments, which involve bulk treating of variable wood species (hardwoods and softwoods) to be used to construct crates and pallets, it is found that installation of a reflective/insulation liner attached to the chamber walls of the RF kiln vessel helps prevent significant thermal heat energy losses during the phytosanitary heating cycle by RFP treatment technology or could equally benefit conventional (non-pressurized dielectric heating) to enable more rapid and cost effective compliance with the ISPM-15 schedule requirements. Numerous materials were tested that can serve as a reflective/insulation liner to control heated wood radiation losses to further reduce RFP operational energy costs and avoid overheating the interior walls of the chamber. An RFP chamber reflective liner installed inside the vessel of the RF unit can prevent significant heat loss to the surrounding steel cylinder by redirecting heat energy back into the wood workload.

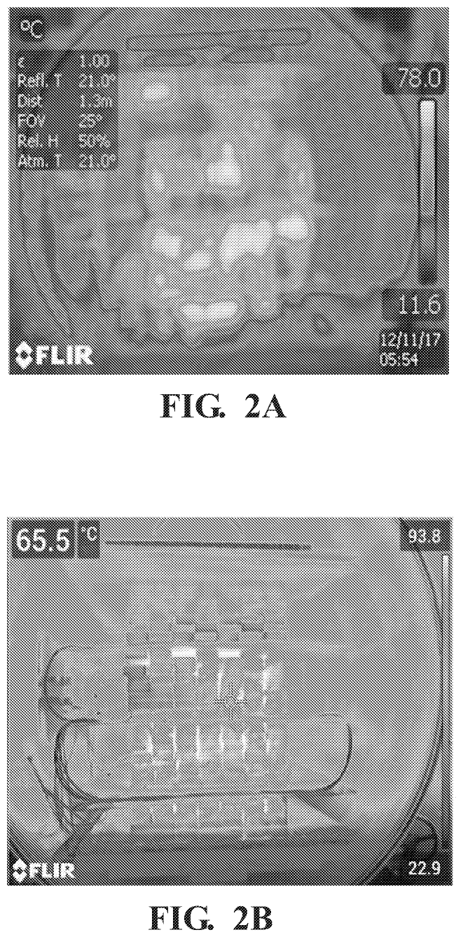

FIGS. 2A-2B are thermal images obtained from a FLIR Model T250 camera. FIG. 2A shows the heating consistency of 4″×6″ cants, whereas FIG. 2B shows the same post-treatment results for wood pallet stringers. FIG. 2B used a higher resolution FLIR model T530 camera. It should be noted that despite differences in appearance, both thermal images, i.e. FIGS. 2A & 2B, have the same emissivity (ε=1.00) value, that takes into account camera calibrations for thermal reading sensitivity.

RF treatment of WPM showed significant thermal energy radiation loss that was absorbed by the more highly conductive steel pressurization vessel (note the thermal imaging difference for the walls of the chamber in the two images in FIGS. 2A-2B, Peak temperature=93.8° C.). The steel wall temperatures during treatment reached 94-96° C., which is a 78° C. increase from the room temperature of 18° C. As a conservative estimation using the material specific heating value of 0.2196 Btu/lb/° C. (mild steel carbon alloy) and RF vessel weight only (equivalent mass of 3,250 lbs), this represents a static loss value of 0.2196 Btu/lb/° C.×78° C.=55,669 Btus. This heat energy loss of 55,669 Btus that could be used to phytosanitize the workload to more quickly attain 60° C. for 1 min as required by ISPM-15 treatment schedule. A RF chamber reflective liner installed inside the vessel of the RF unit can significantly control transient heat loss to the surrounding steel cylinder and redirects heat energy back into the wood workload. Per treatment cycle, this amount of energy is relatively insignificant, but over the life of the equipment, this significantly adds to the RF unit operational cost efficiency and net reductions the carbon footprint.

One of the objectives of this disclosure is to identify materials that can preserve the workload treating heat and avoid energy waste during RF dielectric phytosanitary treatments. First, materials were examined that NASA has used as heat-reflectives in their aerospace vehicles. Heat-reflectivity is one of the major issues for NASA during rocket ignition and traveling through the atmosphere at very high speed, where the spacecraft surfaces reach temperatures over 1600° C. Thermo-shield® is a formulated paint film product inspired by ceramic tiles that NASA uses on its space shuttles. Paint mixed with ceramic compounds has the ability to withstand high temperature exposure and thereby prevent heat damage with penetration to the vehicular system. It contains “millions of microscopically tiny vacuum-filled ceramic bubbles”. Thermo-shield® is advertised or rated as being a highly durable heat-reflective system but is a costly commercial product. The high costs associated with this available product does restrict use for the RFP technology. It requires application of a thin layer to achieve good insulation properties (0.25 mm); as a result of the additions of titanium and aluminum (AL) pigment, it also provides up to 86% sun-heat-reflection. The ceramic bubbles act to prevent heat loss by convection and conduction, since its occurrence depends on material surrounding the object, while a reflective film component preserves the majority of the radiant heat. Technical information of Thermo-shield® paint indicates the following material thermal property values: K=0.054 [W/mK] and R=22.

Another material concept, i.e. Synavax® Heat-reflective High Heat Thermal Insulation, was also examined. Synavax® is a corrosive preventive and moisture resistant paint formulation product, which can be used for steam pipes, tanks, heat exchangers and industrial ovens. It has a clear finish below 77° C. and a white finish above 77° C. Synavax® can withstand temperatures up to 204° C. [2]. It can be applied directly on the metal surface and can be painted over, which indicates that it can be used again to fill in a deteriorated surface. Since it can be applied by brush, roller or sprayed, it can be easily applied to the surface of the complex shapes of RF unit parts. Thermal performance of Synavax® is described as providing a 34.8% decrease in thermal conduction using a three-coat thickness application, where one dried coat is ˜100 microns thick. However, it has a relatively high emissivity of 0.91, while the perfect reflector (e.g. shiny mirror) has an emissivity of zero, and a blackbody (a perfect emitter) has an emissivity of 1.0.

Despite the favorable application properties of these commercial paint type treatments, their material cost factor was determined to be disproportionately high relative to their performance properties for RF treating cylinder applications. As an alternative, a lower cost 100% AL pigmented paint formulation was identified, i.e. Henry® 555 Fibered Aluminum Roof Coat. A thin film of this paint has a limited R-value and is used as a reflectivor to act as a protective shield on the door and end cylinder curvatures. The surfaces were observed to experience lesser amounts of transient heat transfer and require less rigorous thermal shield protection. Henry 555 is formulated with 9% by weight AL content, is commonly used in metal roof applications and is rated to have an effective 56% solar reflectance.

Reflective coatings work well using one or combination of the following components: silica or ceramic microspheres, pigments that are able to reflect the heat radiation and improve reflection of the heat build-up. However, the technical data provided by manufacturers seem to be misleading, presenting the coatings as “insulating”, whereas “They are a radiative barrier minimizing surface heating rather than providing an optimal insulating layer that reduces the potential of long-term conductive heat flow. These paints can lead to a reduction of exterior surface temperatures”. Given some limitations of thin paint films to serve or control conductive heat transfers for an effective RF cylinder liner, further efforts were undertaken to study low cost but effective material options having enhanced insulation properties that can provide sustained heat transfer performance over an extended treating time exposure period.

One such material that was studied included Reflectix® which combines both reflective and insulation properties. This product consists of two outer layers of polymeric type of film that reflect 96% of radiant heat. The heat resistant film consists of two internal layers or sheets of polyethylene bubbles bonded together with a maximum material thickness of 5/16″. Two core layers of insulating bubbles resist conductive heat flow and the double constructed layer of polyethylene gives Reflectix® relatively high strength reliability. The air bubble construction provides an R value equal to 3.0; the air inside the small bubble pockets helps resist against the temperature loss, while the double-sided reflective (aluminum foil) layer reflects the radiant heat back to the RF chamber space loaded with SWPM. This commercial insulator was cost effective and potentially easy for installation either on a flat or single curved surface of a cylinder. This material option when compared to paint application lacks the immediate ability for coverages to any irregular surface and incurs added installation challenges due to internal vessel instrumentation or other equipment operational features. Also, the thermal exposure durability of this product is relatively low rated at only 121° C. maximum temperature resistance. Despite the composite layered construction, this material option was determined to be semi-vulnerable to puncture damages from incidents of loading or unloading of the treatment vessel.

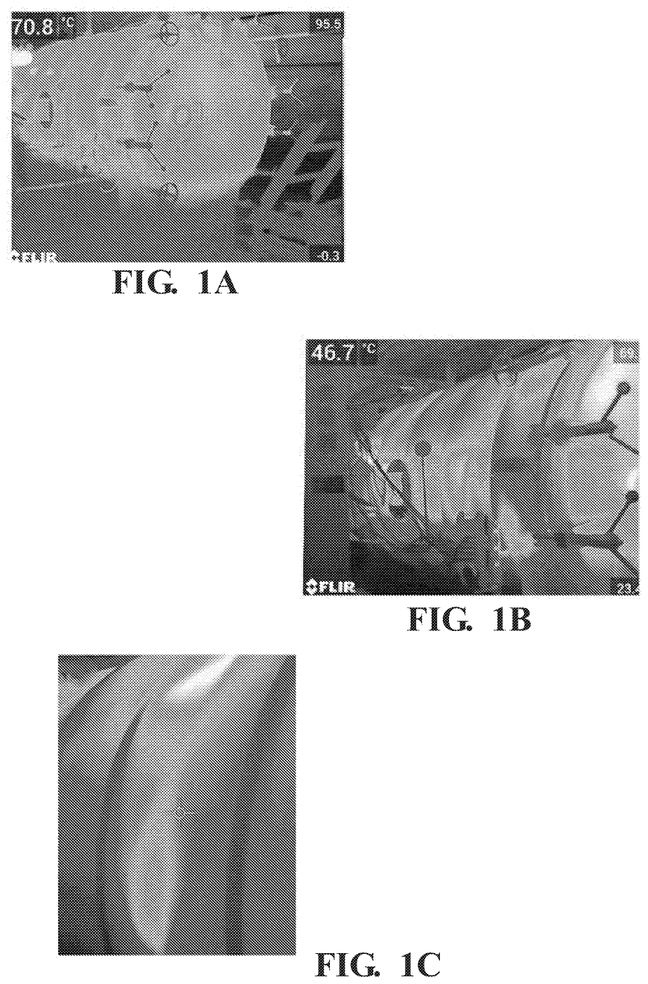

Another material considered was Outdoor Reflectix® that has a 10 times thicker aluminum shell layer (reflects 97% radiant heat) and might be better suited to withstand mechanical punctures. As a commercial product, Reflectix® is available in variable length rolls and sized width. Installations for lining the cylinder with mastic adhesive bonding seams did result in the potential for thermal gaps or heat air to thermal energy transfer losses (refer to FIG. 1C). However, recent information obtained from the manufacturer indicates that this heavier thickness AL shell layered constructed product was removed from the market and was no longer available for test study experimentation.

Additional efforts to examine liner performance properties with higher puncture resistance shifted to testing of another low-cost nonwoven (spun bonded) polypropylene, such as a highly flexible type of a cloth fabric constructed with a reflective outer e.g. anodized AL-metal surface. This material is 17-mm thick with toughened fabric and is commercially produced by Energy Solution (RB Fabric™). This product does not readily yield to puncture or tearing and has a thermal irradiation reflection that approaches 95% efficiency e.g. closely rated performance to Outdoor Reflectix®. During physical testing, this fabric appeared to control or provide heat transfer similar to as provided by the bubble type of insulation treatment. However, a performance defect limitation was identified as the anodized layering was moderately sensitive to abrasive wear and mechanical damage. Overall, this material option is both light weight and provides a good to high thermal heat exposure resistance. RB Fabric was developed and extensively used for higher insulation performance clothing apparel and window curtain applications.

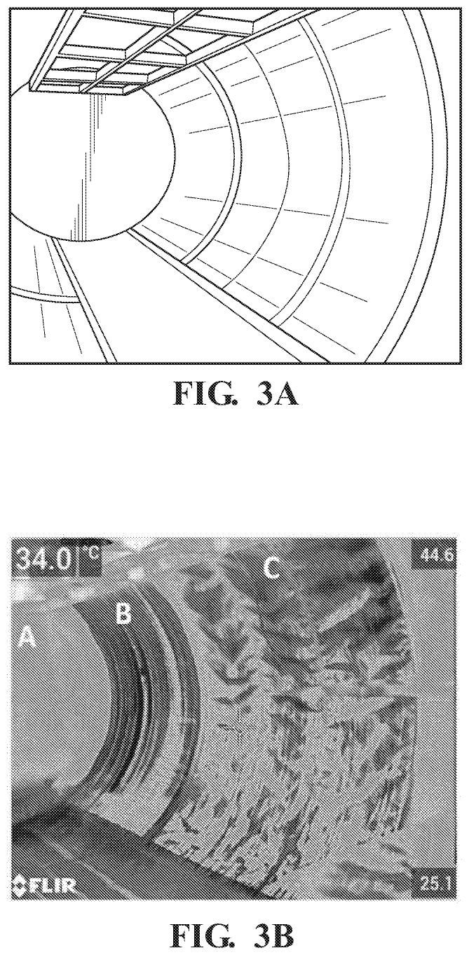

In order to reduce thermal energy transfer to the highly conductive steel kiln cylinder vessel, an Energy Efficient Solutions® RB Fabric™ reflective fabric 17-mil anodized thin film aluminum-coated polypropylene and Reflectix® double-sided aluminum foil with bubble plastic core reflective lining material was attached to the interior wall of the RF chamber vessel. These materials were attached to the inner side of the cylinder using a simple double adhesion attachment (duct) tape. The back wall of the cylinder vessel and front door were painted with Henry® 555 Fibered Aluminum Roof Coating paint. FIG. 3A shows a drawing of a RF chamber covered with these reflective liners. Probes were attached for measuring temperatures on the vessel wall, which were not covered in reflective liner, as a reference. Using these materials, temperatures were monitored at the same locations on the vessel walls, and also on the opposite (exterior) side of the cylinder wall as well. FIG. 3B is a FLIR thermal image of reflective liners inside the chamber in comparison to the image of areas covered with reflective paint and not covered with the experimental insulation material. This thermal image of reflective liners was taken after RF phytosanitation treatment of white ash pallet decking boards. In FIG. 3B, A represents an area of steel vessel covered with reflective AL-pigmented paint, B represents Reflectix® double-sided aluminum foil with bubble plastic core reflective liner, and C represents Energy Efficient Solutions® RB Fabric™ reflective fabric. The differences in temperatures between areas of the workload are pronounced; the area without the liner shows much higher temperatures due to absorption of the dielectric heat produced by the RF unit, while the area (i.e. A, B or C) covered with either of the reflective liners significantly reflected the heat. The area covered with reflective paint alone also shows significant concentration of heat, while the reflective fabric and Reflectix® reflective/insulation liners redirected more heat towards the interior of the chamber.

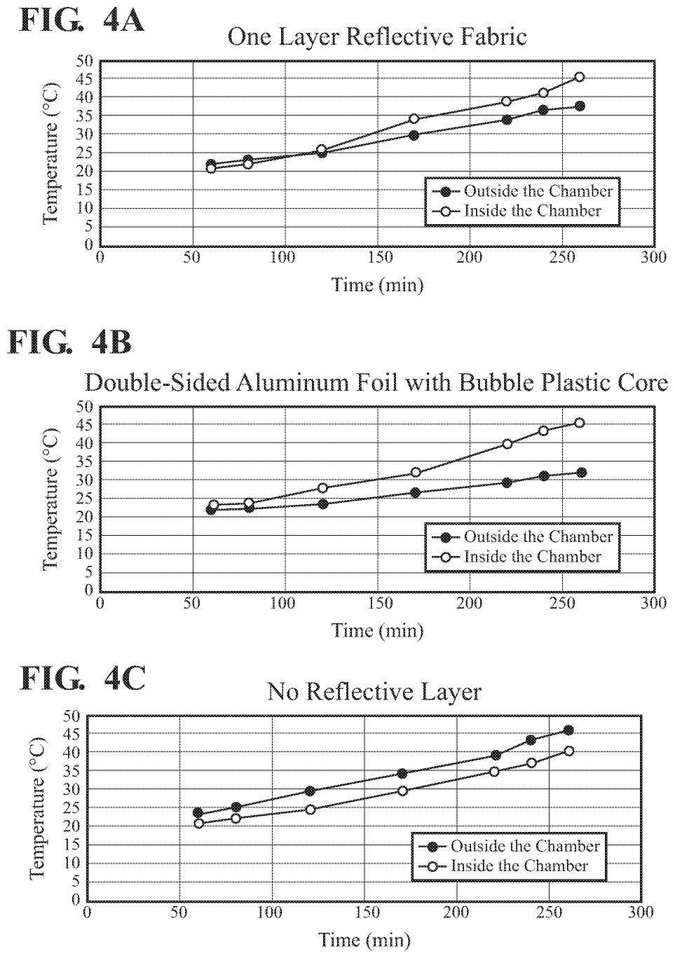

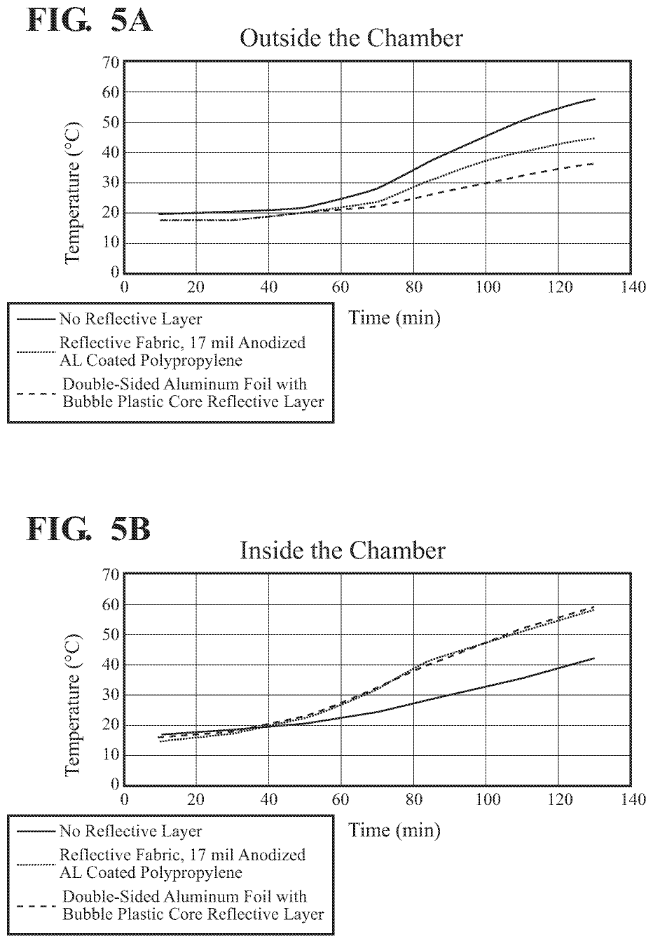

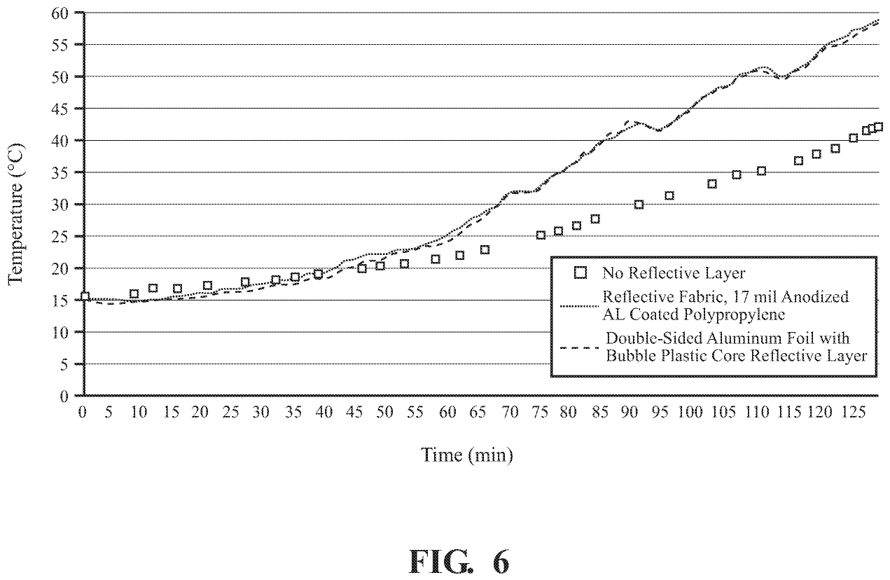

Referring to FIGS. 4A-4C, temperatures on the outer wall of the cylinder stayed well below the peak temperatures noted on the opposite (interior) side of the steel vessel. As shown in FIGS. 5A, 5B and 6, temperatures for the area without the liner inside the vessel during the treatment were lower than temperatures for areas covered with reflective liner. The temperature was lower on the inner wall of the cylinder for the part not covered with any of the liners. For the part covered with one of the liners, the heat was reflected back to the vessel, which also resulted in higher temperature readings on the interior walls of the chamber. For the part without the liner, the heat was absorbed by the steel walls and radiated out of the vessel, so the heat was “drained” to the outside resulting in the lower temperature on the interior wall not covered with the liner. The liner reflected the heat and redirected it back into the vessel to be absorbed by the workload, while the area of the cylinder without the liner absorbed heat into the steel cylinder and allowed it to radiate to the outside of the unit. Application of the reflective liner inside the kiln, therefore, reduces thermal energy transfer to the steel material of the cylinder and lowers the observed heat loss. A reflective vessel liner acts to redirect thermal energy losses from highly conductive steel to the wood materials being treated. Simple insulation of an outer boundary layer would only partially control transient heat losses, while the liner inside the vessel transfers the heat to the commodity, thus converting this thermal energy into a faster commodity heating rate and improved temperature uniformity.

After a series of tests using Reflectix® insulation in the RF unit, it was learned that this insulation is resistant to temperature damage that might occur during treatment conditions after repeated treatment cycles; however, after a few trials the material started to peel off the interior wall, even though it was installed with a double-sided adhesive tape. The method of installation of the reflective insulation liner will depend on the characteristics of the material and the life of the material. A good performing chamber vessel liner should be durable, tear-resistant, light-weight (for example, Al would be a better material than iron/steel), inexpensive, and flexible/bendable to facilitate installation and damage replacement removal from the oven. It is recommended that the liner be able to withstand temperatures of a minimum of 100° C. exposures to a higher 125° C. thermal durability and potential corrosive conditions in the presence of condensed moisture. Additionally, it needs to be resistant to acidic conditions, e.g. moisture with tannic acid released from oak material that is highly acidic, such as a pH in the 3.0 to 3.5 range. From our testing of several materials, some are more durable than others, but all of them, after repeated tests, showed signs of wear. Hence, the chosen liner will need to be easily replaced or refurbished. Attaching the liner to the cylinder wall needs to be performed in a manner that it can easily be removed, and not allow moisture to condense between the liner and the steel cylinder. However, usually the tighter the attachment of the liner to the steel, the more difficult it is to remove for replacement. Also, changing the liner periodically will add to the cost of installation and maintenance time. Hence, the material chosen for this purpose will need to balance the life of the liner material with its performance in reflecting as much heat as possible back to the workload.

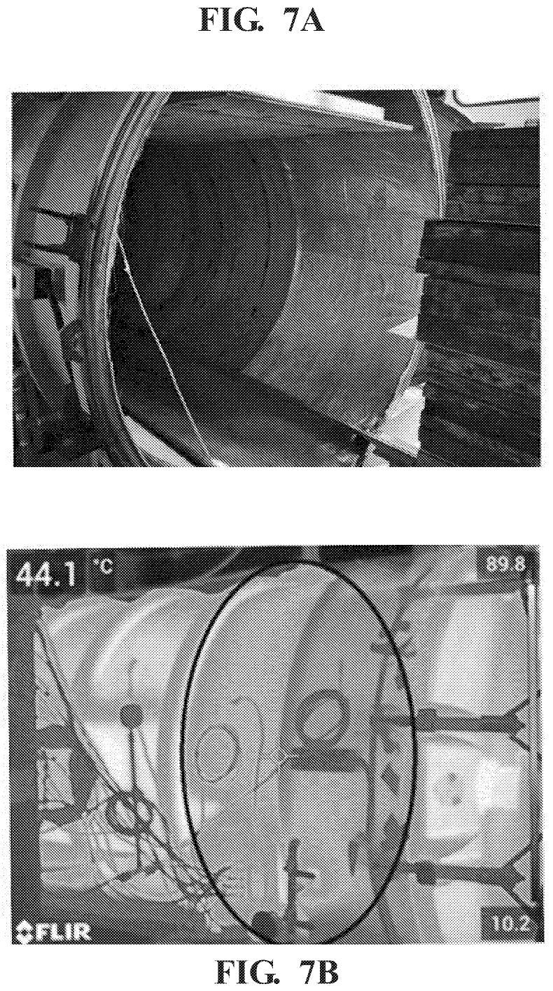

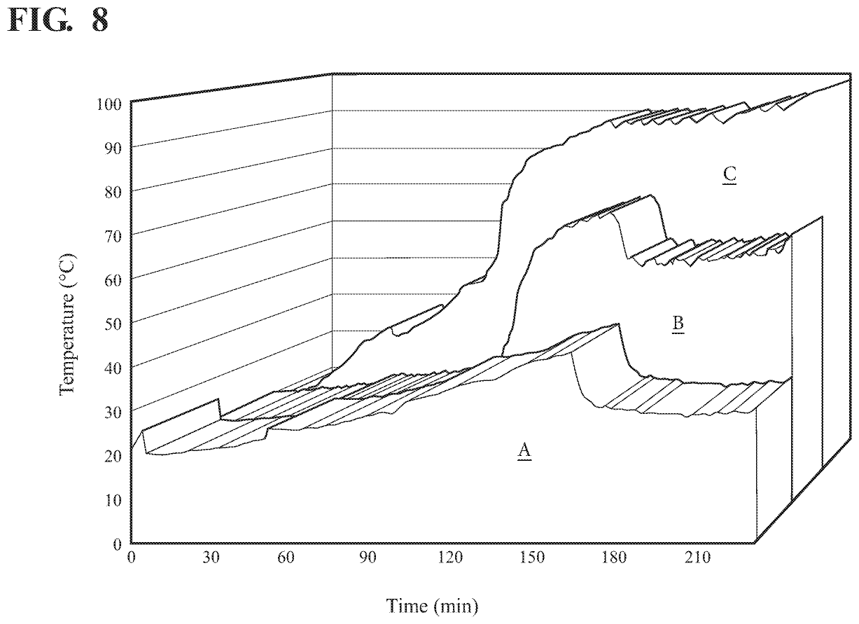

In another experiment, as shown in FIG. 7A, a BISCO® RF-120 Silicone Foam Cast was applied on an Aluminized Fabric reflective/insulation liner to the steel interior walls of the RF chamber. One side of the liner was covered with aluminum foil laminate serving as a reflective layer, while the opposite side of the 5 mm thick foam material (K=0.06 [W/mK] (recommended use at temperatures up to 200° C.)) was covered with adhesive to allow for convenient installation in the RF chamber. Adhesive seals the steel from moisture condensate, preventing it from developing rust and protecting the chamber from deterioration. FIG. 7B is a thermal image of the RF unit during phytosanitation treatment. It should be noted that part of the chamber with installed silicone foam liner inside the RF kiln is marked with the black oval shape. Thermal images (FIG. 7B) and readings, shown in FIG. 8, of the temperature sensors installed on the exterior and interior walls of the kiln insulated with silicone foam and composite constructed reflective liner clearly show its significant heat preserving properties. In FIG. 8, graph A depicts the temperatures observed on the exterior wall of the chamber that is insulated inside with double sided aluminum bubble plastic core liner, graph B depicts the temperatures observed on the exterior wall of the chamber that is insulated inside with silicone foam reflective liner and graph C depicts the temperatures observed on the interior wall of the RF steel chamber. Temperature on the exterior wall of the kiln chamber of the insulated part was 33.9° C. lower than on the part that was covered with a 3-layer reflective air-bubble insulation liner. The temperature on the unshielded interior surface of the chamber vessel increased to 97.9° C. in contrast to 31.1° C. on the exterior wall of the chamber where part of the vessel was insulated by the silicone foam reflective liner. Significant thermal energy radiation was observed in areas not covered with the insulation reflective liner. The 3-layer reflective air-bubble insulation preserved heat loss when the temperature of the interior/exterior wall of the chamber was 65.5/98.4° C.

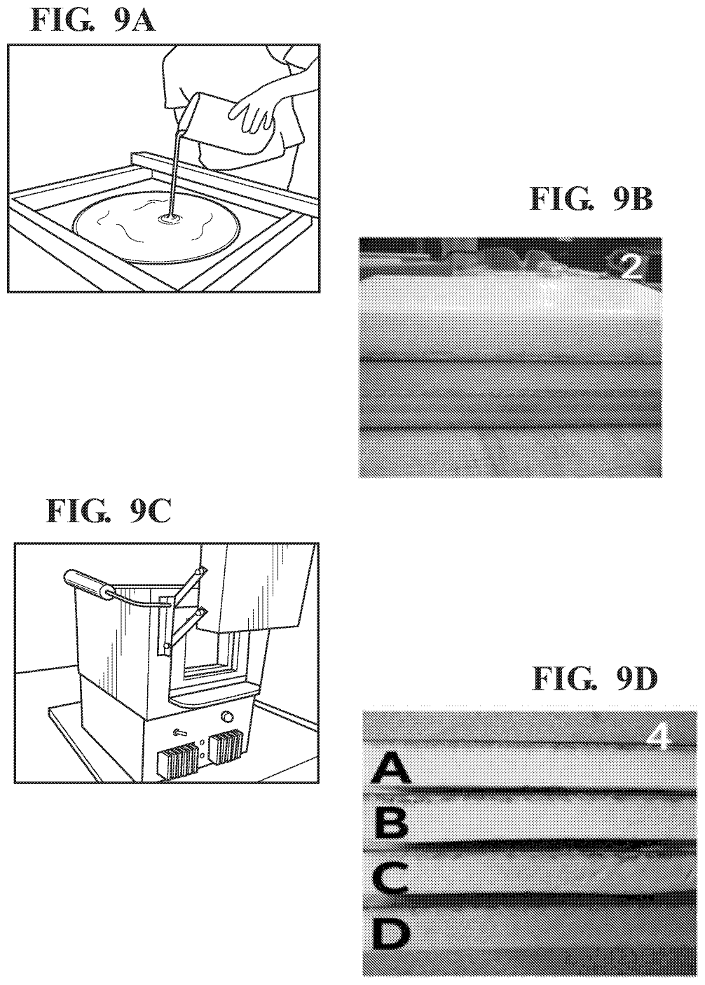

One insulating material that seemed promising for resisting time induced deterioration was Sicomin® PB 250 DM 02, which is a closed-cell type of foaming epoxy system with thermal conductivity K=0.065 [W/mK] and density of 250 kg/m³. This highly durable material needs to be casted on the surface and is designed to bond with the base material permanently. In order to not alter our RF cylinder permanently for the sake of the test, we envisioned a mechanical interlock design with sectional steel plate panel cylinder coverage system. Replaceable steel panels as interlocking sections are held in place by latex rubber double-sided magnetic sheathing that can be easily peeled back for plate section removal. Hence, we casted a 1″ thick layer of the foaming epoxy resin on the light gauge steel plate and investigated its properties. According to the product specification, if the proper curing procedure is performed directly after casting, it should withstand temperatures up to 95° C. However, combining the resin with an enhanced hardener improves its temperature resistance to 129° C. We performed additional temperature resistance tests to examine its thickness shrinkage and mass loss of this material during various temperature conditions (100° C., 130° C., 150° C. for 30 min). We learned that the mass loss did not depend significantly on the temperature the material was subjected to. For 100° C. it was only 0.07% and for other temperatures of treatment we found the same value of 0.13% mass loss. The observed thickness shrinkage however was 1.8% for 100° C. treatment, 0.9% for 130° C., and 6.3% for 150° C. For the sample treated for 30 min in sequence of 100° C., then 130° C. and 150° C. (total treatment time of 90 min), the thickness shrinkage was significantly lower than for the samples treated immediately at 150° C. (1.6%). FIG. 9D shows images (i.e. A, B, C and D) of the samples after the treatment in: A—100° C., B—130° C., C—150° C. for 30 min, and D—100° C., 130° C., 150° C. for the total treatment time of 90 min. These results indicate that after the proper curing of the material, resistance is improved for higher temperature conditions. However, visual examination of the samples revealed temperature related deterioration in the form of partial delamination from the steel sectional plate material.

If the panels are to be connected to the tab, the material should also have good machinability. The Sicomin® epoxy foam had fairly good machinability. However, the epoxy foaming resin did not adhere to the steel panel sufficiently (FIG. 9B) during the treatment cycle. We observed that the cured epoxy foam began to debond from the steel panel when exposed to sustained temperatures over 130-150° C. Use of this casting system may require a change in the surface activation energy to improve adhesion to overcome in-service steel thermal expansion and dimensional changes, as the liquid resin cures into the expanded foam layer to provide long term performance. Therefore, there are performance issues still left to be investigated and resolved for this type of insulation layer to work adequately and at low cost per square feet of the area covered inside the RF cylinder.

Sicomin® also provides the PB 360 GS/DM 07 system, which can be processed by casting or spraying. The set contains a very fast hardener that allows it to be sprayed directly on the surface in needed areas. It has a thermal resistance up to 100° C., good adhesion to many types of materials, very low water absorption, and a density of 360 kg/m³; however, it requires professional equipment, e.g., heatable low-pressure mix-metering, which raises the question of cost effectiveness.

In one preferred version of the present invention, a majority of the inner surface of the chamber is covered by a thermal barrier liner, where the liner includes a heat-reflective face and an insulation backing layer installed and/or attached to highly conductive steel surfaces of the pressurized RF treating chamber. Covering more of the chamber surface is generally preferred and in some examples 75% or more of the transient or heat transmission surface area is covered with the composite designed system as a thermal barrier, while other parts may be untreated or may be painted with heat-reflective paint. In one example, the heat-reflective face is aluminum foil or aluminum fabric, such as aluminum anodized polyester fabric. Preferably, the heat-reflective inner face has a heat reflectivity of at least 90%, or even as high as least 95%. This reflectivity may be defined in the IR range. The insulation layer may be an insulating foam, such as silicone foam, epoxy foam or polyamide foam, having an thermal conductivity of less than 0.07 W/mK or less than 0.05 W/mK. The heat-reflective face should be tightly bonded to the insulating foam. Preferably, the installation procedure should be effective to minimize air gaps between the insulation layer to the installation surface of the steel chamber. This means that air gaps are reduced as much as practically possible in field application conditions to exclude an interface where liquid water to gaseous vapor may accumulate.

It is preferred that the insulation layer not absorb and retain moisture, and that the heat-reflective inner face resists the passage of moisture from the active treatment space into the insulation. The edges and seams should be properly sealed to avoid moisture transport. In some examples, the insulation layer has a moisture retention of less than 5% weight gain when exposed to moisture from RF operating to treating conditions for a 24 hour period. To reduce moisture transport, the heat-reflective face may or should be moisture impermeable, defined as having a permeability rating of 0.1 perm or less. The effectiveness of a material to control diffusion is measured by its permeability or perms. A perm is defined as the ability to pass one grain of water vapor per hour through one square foot of flat material at one inch of mercury (gr/h*ft²*in·Hg). One grain of water is 1/7000 of a pound or 0.0022 ounces of water. Many building materials are tested to measure permeability, the result of this test is perm rating. The higher the number, the more readily water vapor (in the gaseous state) can diffuse through the material. A perm rating of less than 0.1 is considered a vapor barrier; perm between 0.1 and 1 is considered a vapor retarder; a perm between 1 and 10 is semi-permeable; and a perm rating greater than 10 is considered permeable.

It is also preferred that the liner can tolerate acidic conditions, such as may result during the treatment of oak. The liner may have an acid tolerance to a pH level of 3.0 or of 3.5, which is defined such that the liner (thermal barrier design) should not chemically deteriorate significantly under operations where chamber treating conditions in which this pH level is present.

Insulating the RF chamber vessel might result in significant heat and energy savings, but in addition, it is also advantageous to insulate the wood load itself to provide a more even distribution of heat in the workload and to reduce the treatment duration. Therefore, we tested various materials for insulating the wood load by installing it under and/or on the top of the workload. While flexible insulation material might be more convenient for applications of complex shapes of the system components, rigid material is superior for insulation of the workload, as it usually has better compression and impact strength characteristics. The system of cast epoxy offers a higher degree of flexibility to process a variable thickness insulator with a prescribed R-value to match the cylinder treating requirements as an optimized thermal shield.

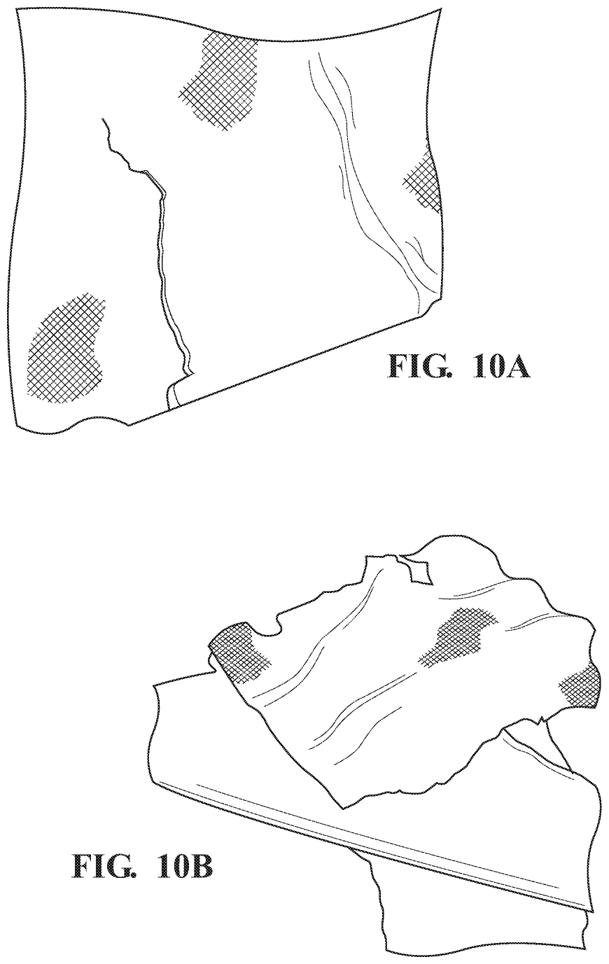

In another experimental trial, we tested the material concept of using Manton® cork liner installed on part of the vessel. We also examined the cork usability as an insulator of the wood load. Cork is the bark of the cork oak (Quercus suber L.) and is composed of a honeycomb of microscopic cells filled with an air-like gas that makes it a good insulator. It is very lightweight, since 50% of its volume is air. It is elastic, compressible, and resistant to abrasion and impact and has a content of suberin and ceroids in the cell walls that makes cork impermeable to liquids and gases. Although it has good natural insulation properties (e.g. thermal conductivity K=0.043 [W/mK]), investigation of the liner revealed that it significantly absorbed water and water vapor after treatment cycle depressurization. The temperature measured on the outer side of the cylinder wall was 31.1° C. in the part of the unit that was not insulated by cork liner, while the temperature on the outer wall of the cylinder insulated with the cork liner was 27.9° C., which is only a difference of 3.2° C. As shown in FIGS. 10A and 10B, ¼″ thick 100% natural cork sheet material installed on the inner wall of the RF chamber and on top of the wood load, “sponged up” rapid swelling due to moisture released by the wood load during the dielectric treatment cycle and dramatically lost its durability to provide sustained thermal resistance. This illustrates that although cork can resist moisture absorption under atmospheric conditions, it could not withstand the moisture during operating conditions of added vacuum and pressure conditions. Therefore, the cork material is not suitable for the RF internal chamber or load insulation.

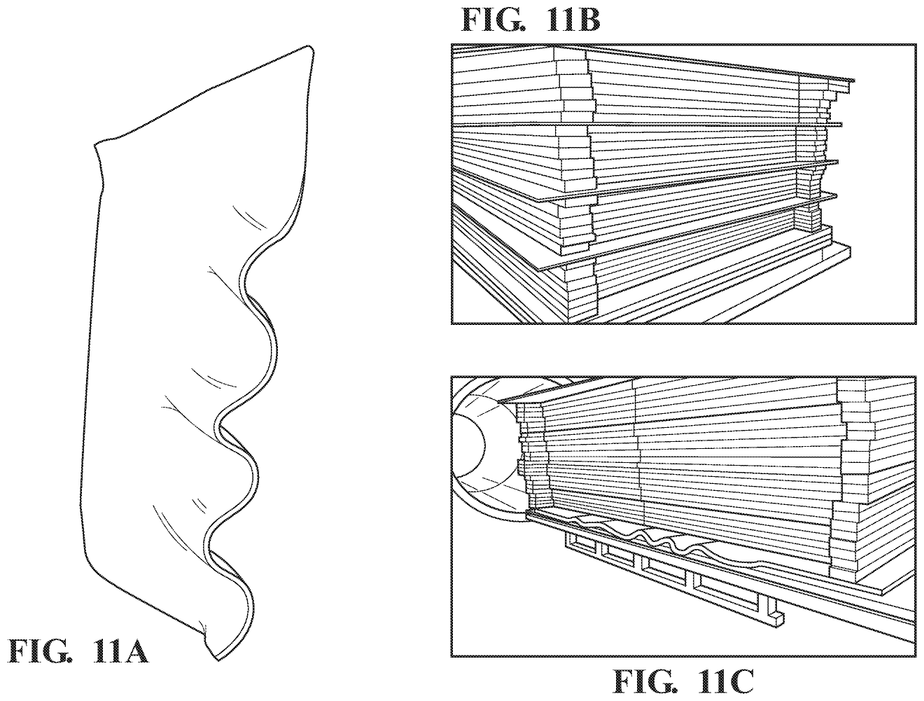

FOAMULAR® 250 of 2-inch thickness×4 ft.×8 ft. R-10 Scored Squared Edge Rigid Foam Board is a light-weight closed-cell polystyrene foam (XPS) board panel insulation that provides a high R-value of 5 per inch of material thickness and thermal conductivity of K=0.02 [W/mK]. This low cost commercially extruded material is 100% moisture proof with good chemical resistance and is easily machinable by cutting and sawing. It provides good durability and is easy to handle and install on the top and under the load of wood material. Foam Board Panels can be layered to provide more insulation and adjust the height of the wood load. Rigid foam boards provide continuous insulation over the wood and are resistant to damage caused by the press compressing the material from the top side of the load. The foam that we tested is characterized by a compressive strength of 172 kPa, min (25 psi), and it showed minimal deformation after compressing by the RF unit press; however, there are products on the market that have compressive strength of 690 kPa, min (100 psi). While polystyrene foam is an excellent, easy to use and cost-effective insulation, it also brings some challenges if it were to be used as a liner material. As it is a rigid foam, installation in the cylindrical chamber would require creating parallel partial cuts of the material in its thickness so that it can conform to the curvature of the cylinder. This could be overcome if the RF unit vessel were designed as a rectangular/square versus a round cylinder. However, this polymeric material has a maximum rated service temperature of only 74° C. RF operations often exceed this temperature threshold at the top section of the load in direct contact with the insulation, which, as shown in FIGS. 11A-11C, causes deformation of the insulation and the loss of its insulating properties. FOAMGLAS® ONE™ Insulation is a lightweight, rigid material composed of millions of completely sealed glass cells and is designed for hot oil and hot asphalt storage tanks. It therefore has a service temperature up to 482° C. As per the manufacturer, it is impermeable to heated water and elevated temperature water vapor, corrosion/chemical resistant, and has high compression strength of 620 kPa (90 lb./in²).

We also tested a Rothco® European Surplus Style 90% wool blanket that is a replica of popular Italian army type wool blankets, which are naturally fire retardant, durable, rugged and designed for maximum retained heat insulation. Sheep wool is a very good insulator due to the crimped nature of wool fibers, which form millions of tiny air pockets that trap air and provide a thermal barrier preserving heat. Sheep wool insulation has a thermal conductivity between 0.035-0.04 [W/mK], whereas typical mineral wool has a thermal conductivity of 0.044 [W/mK]. Due to the high nitrogen content of natural sheep's wool, it is fire resistant and because it is a natural “keratin” biopolymeric material it is sustainable. Sheep wool insulation has an R-value of approximately 3.5 to 3.8 per inch of material thickness. The insulation properties of the wool blanket will depend on its thickness and density. Compared with other synthetic fabrics, woven wool fibers as the insulation material exhibit compatible permittivity properties for this application when exposed to the RF dielectric electromagnetic field (EMF). Other synthetic materials such as manufactured polymeric fibers can have negative impact in the form of systematic EMF (operation wavelength) reflections. These reflections cause undesirable slow to reduced thermal elevation of the treated wood workload.

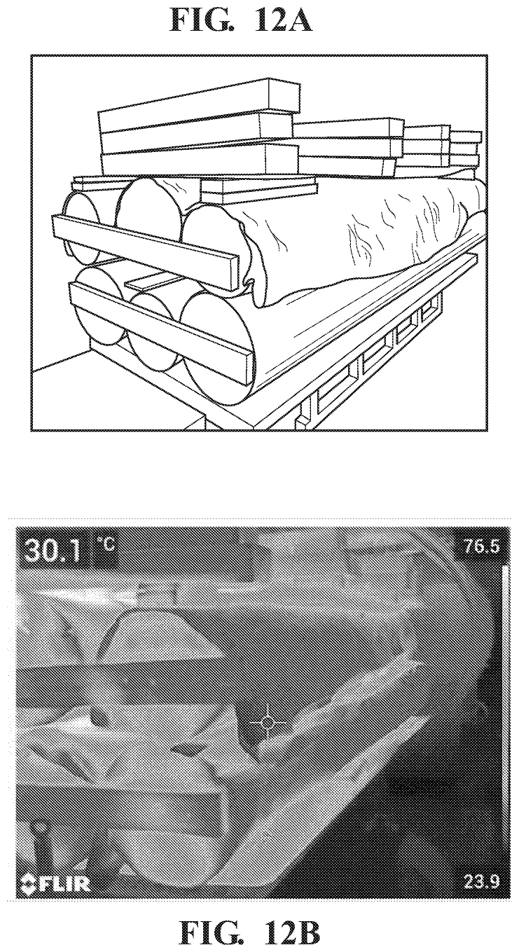

The concept of insulating the wood load with wool blankets arose when we were searching for a way to insulate a load of Eastern white pine logs and we were experiencing significant air gaps between the wood material and flat electrodes (see FIG. 12a). Wool blankets are durable and very flexible, so we covered 3 out of 6 treated logs to examine its insulation properties. The average temperature at the end of experiment recorded by probes located in the logs not covered with wool blankets was 46° C., while the average temperature recorded by probes inserted in the logs insulated by wool blankets was 53° C.; this resulted in an average decrease in recorded temperatures of 7° C. or 13.2%, proving its insulation utility for this type of treatment. The wool blanket absorbed a significant amount of water, but we did not observe a visible disintegration or loss of durability of the material. Natural wool blanketing is known to retain its inherent insulation properties even after reaching saturation of absorbed moisture.

Superwool® blankets is another example of this kind of commercial product with an extremely high temperature durability resistance up to 1200° C. and density of 160 kg/m3 and have a similar thermal conductivity K=0.04 [W/mK]. These blankets have generally acceptable set of permittivity properties that are largely suitably matched for EMF exposures as an insulation wool for dielectric heating applications. It has a good resistance for tearing and would presumably absorb significant amounts of water during the treatment, but it is uncertain whether this alternatively type of blended wool might experience a decrease in its overall insulation quality.

There are many closed cell (CC) insulation materials on the market as opposed to lower insulation quality performance open cell foams. General Plastics R-9300 Structural Continuous Insulation Series is a high-density rigid cellular polyurethane custom manufactured and supplied as CC material with expanded densities ranging from 320 to 641 kg/m³ with compressive strength varying from 2,400 to 14,500 psi and thermal durability temperatures up to 119° C. It combines high compressive strength with limited deflection and good thermal insulation. This polymeric closed-cell material does not absorb water and can function to restrict moisture movements to control adverse steel corrosion to service protection of the critical RF pressurized treatment cylinder.

As a result of this disclosure, significant treatment cost savings can be realized by reducing energy consumption and providing overall improvements in the processing efficiency of RF treatment in compliance with ISPM-15 standard requirements or for other applications using RF to apply heat. Adding the reflective liner lowered the temperature of the vessel, indicating that some amount of heat was preserved and kept from radiating into the vessel walls, improving the heating rate for the wood commodity instead. This positively affects the treatment time for the workload without having to increase the power density to meet the treatment schedule of ISPM-15 or just to reduce energy costs during treatment.

An embodiment of the present invention with the liner will now be described with reference to FIG. 13. FIG. 13 shows a front view showing inside an exemplary RF chamber. FIG. 13 is a schematic view of an exemplary layout of a radio frequency system for RF dielectric treatment of the wood packaging materials (WPMs). In one embodiment, as shown in FIG. 13, the system arrangement includes a sealed chamber 10, used as a pressurization treating cylinder or treatment retort, a pressurization system 11, e.g. an air supply pump for retort pressurization (not shown), a RF operational cooling system (not shown), a RF (3-30 MHz) electromagnetic input power generator (oscillator or other) 14, with a suitable integrated PLC control system as the functional mechanism for applied power density to regulate targeted WPM heating rates, and an infeed/outfeed track loader (not shown) for loading and unloading of the workload. Overall, the cooling system of higher power RF heating units must be suited for rapid cycle sanitization, e.g., those that run with applied operational power below 30-50 kW, which may optionally include only an air-induction fan system for cooling to dissipate excess RF tube heat.

The chamber 10 shown in the center region of the layout may be an adequate construction cylinder or box-shaped design. In one example, the chamber 10 is the type of chambers used for vacuum with moisture drying treatments of wood, in the form of sawn lumber and timbers. Our design was specifically modified to allow or enable chamber retort pressurization. The chamber 10 can include either a manual or hydraulic sealable door which can be freely swung open or closed to facilitate loading/unloading the volumetric batches of WPM.

In one embodiment, the system has a 3-phase electrical source of 480 or optional 600 volts and a total input power of 125-150 amps at 480 volts, supplied by the service alternating current (voltage with power input) transformer.

In one embodiment, the system includes a cooling system having a cooling capacity of 159960 kcal/h or higher. The cooling system may be an evaporative cooling system comprised of stainless-steel cabinets, heat exchangers, water circulation pumps and exhaust fans.

As shown in FIG. 13, the chamber 10 includes two primary electrodes including a retractable compression electrode plate 20 as the top electrode and a ground electrode 22. An inner surface of the chamber 10 is covered with the reflective liner 50. In non-limiting examples, the reflective liner 50 may be attached with the inner surface using an adhesive; the reflective liner 50 may be fastened or disposed in slots designed for holding the reflective liner 50 in the inner surface of the chamber 10.

The retractable compression top electrode plate 20 is lowered or retracted by air cylinders between the loading and the unloading of the workload. The bottom ground electrode is in a position fixed inside the lower portion of the retort 10. As the workload is fed into the chamber 10 by the infeed/outfeed track as the workload transport loader 16, the volumetric workload is placed on the transport table 24 and positioned between the top electrode 20 and the bottom ground electrode 22. The top electrode applies a download load pressing down onto the workload to assist or remove the air gaps between the top electrode and the lower ground electrode.

Additional secondary electrodes may be used to improve the energy field distribution depending on the depth of the workload. Secondary electrodes may be statically placed between the built-up rows of WPM to be treated and applied as a batch treatment. The secondary electrodes are manually removed after the workload is effectively removed from the cylinder. In an alternative embodiment, instead of secondary electrodes, the top flat electrode may be modified with a winged electrode design arrangement. The top flat electrode 30 may include electrode plate wings, e.g., along the entire perimeter of the flat electrode plate 30, including two ends and two sides. FIG. 14 is a cross-sectional view showing the reflective liner 50, insulation liner 60, three secondary electrodes 32, 34, 36 attached to the flat electrode plate 30, one at each end and one of the parallel sides of the electrode 30, facing the bottom ground electrode 22.

FIG. 15 is an exploded view of detail A shown in FIG. 14. It shows the reflective liner 50 covering the inner surface and the insulation liner 60 disposed between the reflective liner 50 and the inner surface 70 of the chamber 10.

The primary electrode pair or secondary electrodes are connected to the RF power input generator 14. The RF generator 14 supplies an alternating current to introduce an electromagnetic field. In one embodiment, the RF generator has a constant or variable power output of 50 kW or with greater heating rate capacities. In one embodiment, an operational electromagnetic dielectric frequency may be in the range of 5 and 30 MHz or other wavelength frequency suitable to achieve the desired depth of penetration for wave energy adsorption to obtain heating uniformity during dielectric electromagnetic treatment of an entire WPM volume. The pressurization system (not shown) provides systematic pressurization of the chamber during the active RF treatment. Just as water evaporates at a higher temperature under an air pressure higher than atmosphere, the pressurization technique of the present disclosure helps to prevent moisture and significant thermal heat energy losses during the phytosanitary heating cycle by RF treatment to more rapidly and cost effectively comply with ISPM treating requirements.

The temperature within the workload may be monitored throughout the treatment. The temperature monitoring may be done by factory-calibrated fiber-optic or other RF compatible temperature sensors. An access port (not shown) on one side of the retort enables running (routing) of the required fiber-optic sensors inside the retort and continuous monitoring of the workload heating coupled to an independent data collection system.

Some exemplary dimensions of a system in accordance with the present disclosure are as follows. In one embodiment, the chamber measures 3-m×1-m×1-m. The volume capacity to be heated as shown is equal to ˜3 cubic meters, although greater capacity workload designs may be built for large-scale commercial treaters. The electrode plates measure roughly 3-m×1-m. The infeed/outfeed track loader measures 4-m×1-m.

An important component of the RF system innovation includes adequate positive pressure control to raise the boiling point of water or otherwise control the conversion of liquid moisture content to a gaseous water vapor phase that results in net moisture content reduction, while also preventing the critical losses of thermal energy needed to rapidly and with desired uniformity elevate the WPM temperatures throughout the bulk volume of the treated load. Energy losses may be reduced by providing a reflective liner on the inner surface of the chamber, which can reflect the thermal radiation from the chamber walls back towards the heated wood material. Adding the insulation liner to the inner surface helps to preserve the remaining energy that is not reflected or transferred by conduction or convection.

There are a number of materials of similar composition used to reflect hot temperatures. Economic feasibility, durability (expected life of the liner) and reflective efficiency of the liner material should be taken under consideration when deciding which reflective material to use, as the choice of materials will define the economic benefits of using a reflective liner in the RF unit.

As will be clear to those of skill in the art, the embodiments of the present invention illustrated and discussed herein may be altered in various ways without departing from the scope or teaching of the present invention. Also, elements and aspects of one embodiment may be combined with elements and aspects of another embodiment. It is the following claims, including all equivalents, which define the scope of the invention.

1. A method of treating wood packaging materials (WPMs) using Radio Frequency (RF) heating, the method comprising the steps of:
providing a RF operating unit including:
a sealable chamber having an inner surface,
a liner covering a majority of the inner surface, the liner having a heat-reflective inner face and an insulation layer between the inner face and the inner surface of the sealable chamber,
a RF generator connected to the chamber for applying RF heating rea ent to the WPM,
a pressurization system for controlling the pressure inside the chamber, loading the chamber with a workload of the WPMs;
applying a pressure to the chamber during the treatment, the pressure being at least 5 psi greater than atmospheric pressure;
treating the WPMs using RF heating until a temperature of the WPMs reaches a predetermined temperature not more than 100° C.; and
maintaining the predetermined temperature for at least 1 minute.

2. The method of treating wood packaging materials in accordance with claim 1, wherein the liner covers at least 75% percent of the entire inner surface of the sealed chamber.

3. The method of treating wood packaging materials in accordance with claim 1, wherein the heat-reflective inner face of the liner is aluminum foil, aluminum fabric, or aluminum anodized polyester fabric having a heat reflectivity of at least 90%.

4. The method of treating wood packaging materials in accordance with claim 1, wherein the insulation layer is silicone foam or polyamide foam having an thermal conductivity of less than 0.07 W/mK.

5. The method of treating wood packaging materials in accordance with claim 1, wherein there is substantially no air gap between an outer surface of the insulation layer and the inner surface of the chamber.

6. The method of treating wood packaging materials in accordance with claim 1, wherein the insulation layer has a moisture retention of less than 5% weight gain when exposed to moisture for a 24 hour period.

7. The method of treating wood packaging materials in accordance with claim 1, wherein the reflective face is moisture impermeable, having a permeability rating of 0.1 perm or less.

8. The method of treating wood packaging materials in accordance with claim 1, further comprising placing an insulation layer on the top and/or under the WPM prior to the pressure applying step, wherein the insulation layer is wool having a thickness of at least 0.1 inch.

9. The method of treating wood packaging materials in accordance with claim 1, wherein the liner has an acid tolerance to a pH level of 3.0.

10. The method of treating wood packaging materials in accordance with claim 1, wherein the predetermined temperature is not less than 60° C. and is not more than a maximum temperature of 90° C.

11. The method of treating wood packaging materials in accordance with claim 1, wherein the predetermined temperature is maintained for not longer than a period of 5 minutes.

12. The method of treating wood packaging materials in accordance with claim 1, wherein the step of applying of the pressure to the chamber comprises maintaining the chamber at a first pressure during a first period and changing the pressure in the chamber to a second pressure after the first period, the first pressure being approximately atmospheric pressure and the second pressure being at least 5 psi greater than atmospheric pressure.

13. The method of treating wood packaging materials in accordance with claim 12, wherein the first period is defined by a temperature threshold, the first period ending when the temperature of at least some of the WPMs reach a temperature threshold in the range of approximately 30° C. to approximately 60° C.

14. The method of treating wood packaging materials in accordance with claim 1, further comprising depressurizing the chamber after reaching at least 60° C. with a 1-minute hold time.

15. The method of treating wood packaging materials in accordance with claim 14, wherein the depressurizing of the chamber is at a rate of decreased pressure of 2-4 psi per minute.

16. The method of treating wood packaging materials in accordance with claim 1, wherein the heating treatment is at a constant rate or at a ramping rate.
